(19) United States
(12) Patent Application Publication
(10) Pub. No.: US 2022/0386900 A1
(43) Pub. Date: Dec. 8, 2022

(54) Rehabilitation Support System and Rehabilitation Support

(71) Applicant: Nippon Telegraph and Telephone Corporation, Tokyo (JP)

(72) Inventors: Rieko Sato, Tokyo (JP); Takayuki Ogasawara, Tokyo (JP)

(73) Assignees: Nippon Telegraph and Telephone Corporation, Tokyo (JP); Nippon Telegraph and Telephone Corporation, Tokyo (JP)

(21) Appl. No.: 17/776,845

(22) PCT Filed: Nov. 14, 2019
(86) PCT No.: PCT/JP2019/044673

§ 371 Date: May 13, 2022

(51) Int. Cl. A61B 5/11; A61B 5/00; A61B 5/024

(52) U.S. Cl. CPC A61B 5/1118; A61B 5/744; A61B 5/02438; A61B 5/1126; A61B 2562/0219; A61B 2505/09

(57) ABSTRACT

A rehabilitation support system includes a data acquisition unit that acquires sensor data including biological information, an activity amount calculation unit that calculates an activity amount accompanying activity of user, based on the acquired sensor data, a cumulative value calculation unit that calculates the cumulative value of the activity amount of the user in the set period, a display processing unit that generates an image item corresponding to the cumulative value, and a second display unit that displays the image item generated by the display processing unit on the display screen, and a plurality of the set periods are included in the period of activity of the user.

This patent application is a national phase filing under section 371 of PCT application no. PCT/JP2019/044673, filed on Nov. 14, 2019, which application is hereby incorporated herein by reference in its entirety.

### TECHNICAL FIELD

The present invention relates to a rehabilitation support system and a rehabilitation support method, and in particular to a rehabilitation support technique for improving motivation of a user performing rehabilitation.

### BACKGROUND

Conventionally, in the field of rehabilitation for recovery of body function, feedback devices and systems in which game elements are incorporated for the purpose of improving the motivation of users such as patients performing rehabilitation (hereinafter, simply referred to as “rehabilitation”) have been proposed (see, for example, NPL 1).

In the conventional rehabilitation support system, the level of achievement of rehabilitation is fed back to each user so that the user's motivation for rehabilitation is increased and the user actively works on the rehabilitation.

When the rehabilitation achievement level is individually fed back to each user, even if multiple users have the same rehabilitation achievement level and the same feedback is given individually to these multiple users, the influence that the feedback has on the user differs for each user. For this reason, the effect of improving motivation to work on rehabilitation may differ for each user.

Also, for example, feedback of the rehabilitation achievement level to a user who has a relatively large physical activity amount accompanying the rehabilitation has a greater encouraging effect on the user. However, the effect of improving motivation for rehabilitation through feedback of the rehabilitation achieved level to a user with a relatively small activity amount may not be so great. Here, “activity amount of the user” means the amount of physical activity accompanying rehabilitation for achieving recovery of body function, and includes, for example, exercise intensity, calories burned, wake-up time, standing time, step count, or the like.

Furthermore, in a conventional rehabilitation support system, the user can know the effort for working on the user's own rehabilitation, but not the user's relative effort in relation to another user. For this reason, it has been difficult to maintain or improve the overall motivation of users working on rehabilitation, including users with a relatively small activity amount.

### CITATION LIST

### Non-Patent Literature

[NPL 1] K. Yoshioka “Development of Art Program for Feeding Back Level of Achievement of Rehabilitation Exercises (2)” Japanese Society for the Science of Design, BULLETIN OF JSSD 2017, pp. 196-197.

### SUMMARY

### Technical Problem

Embodiments of the present invention have been made to solve the above-mentioned problems, and an object of embodiments of the present invention is to improve the motivation of all users to work on rehabilitation.

### Means for Solving the Problem

In order to solve the above-mentioned problems, a rehabilitation support system according to embodiments of the present invention includes: a data acquisition unit configured to acquire sensor data including biological information; a first calculation unit configured to calculate an activity amount accompanying activity of a user, based on the acquired sensor data; a second calculation unit configured to calculate a cumulative value of the activity amount of the user in a set period; a display processing unit configured to generate an image item corresponding to the cumulative amount; and a display unit configured to display, on a display screen, the image item generated by the display processing unit, in which a plurality of the set periods are included in a period of activity of the user.

In order to solve the above-mentioned problems, a rehabilitation support method according to embodiments of the present invention includes: a first step of acquiring sensor data including biological information of a user measured by a sensor; a second step of calculating an activity amount accompanying activity of a user based on the sensor data acquired in the first step; a third step of calculating a cumulative value of the activity amount of the user in a set period; a fourth step of generating an image item corresponding to the cumulative value calculated in the third step; and a fifth step of displaying, on a display screen, the image item generated in the fourth step, in which a plurality of the set periods are included in a period of activity of the user.

### Effects of Embodiments of the Invention

According to embodiments of the present invention, based on the cumulative value of the activity amount of the user in the set period, an image item corresponding to the cumulative value of the activity amount is generated, and is displayed on the display screen, and therefore it is possible to improve the motivation of all users to work on rehabilitation.

### BRIEF DESCRIPTION OF THE DRAWINGS

FIG. 1 is a block diagram illustrating an overview of a rehabilitation support system according to an embodiment of the present invention.

FIG. 2 is a block diagram showing a configuration of a data analysis unit according to the present embodiment.

FIG. 3 is a block diagram showing an example of a computer configuration for realizing the rehabilitation support system according to the present embodiment.

FIG. 4 is a flowchart for describing operations of the rehabilitation support system according to the present embodiment.

FIG. 5 is a block diagram showing a specific configuration example of the rehabilitation support system according to the present embodiment.

FIG. 6 is a sequence diagram illustrating operations of the rehabilitation support system according to the present embodiment.

FIG. 7 is a diagram for illustrating a first display example of a second display unit according to the present embodiment.

FIG. 8 is a diagram for illustrating a first display example of a second display unit according to the present embodiment.

FIG. 9 is a diagram for illustrating a second display example of a second display unit according to the present embodiment.

FIG. 10 is a diagram for illustrating a second display example of a second display unit according to the present embodiment.

FIG. 11 is a diagram for illustrating a third display example of a second display unit according to the present embodiment.

FIG. 12 is a diagram for illustrating a third display example of a second display unit according to the present embodiment.

FIG. 13 is a diagram showing a display example of the first display unit according to the present embodiment.

### DETAILED DESCRIPTION OF ILLUSTRATIVE EMBODIMENTS

Hereinafter, preferred embodiments of the present invention will be described in detail with reference to FIGS. 1 to 13. Also, in the present embodiments, a case where users such as a plurality of patients perform rehabilitation training for recovering motor function in the same time span in a training room or the like of a hospital, a rehabilitation facility, or the like will be described as an example.

### Overview of Rehabilitation Support System

First, an overview of a configuration of a rehabilitation support system 1 according to an embodiment of the present invention will be described with reference to FIG. 1. The rehabilitation support system 1 includes a second display unit (display unit) 16 for sharing and displaying information relating to rehabilitation support among users on a display screen.

The rehabilitation support system 1 acquires sensor data including biological information of each user, which was measured by a sensor such as a wearable sensor worn by each user performing rehabilitation. Also, the rehabilitation support system 1 collects the sensor data acquired for each user at regular intervals, for example, calculates the activity amount of each user, and calculates the cumulative value of the activity amount of each user in a set period.

The rehabilitation support system 1 generates an image item for each user corresponding to the size of the cumulative value of the activity amount based on the cumulative value of the activity amount of each user in the set period, and shares and displays the generated image item on the display screen. In the present embodiment, the training period in which rehabilitation is carried out is constituted by a plurality of the above-described set periods, and the image item is updated according to the size of the cumulative value of the activity amount each time the set period elapses. In this manner, a plurality of users performing rehabilitation can visually keep track of not only their own but also another user's rehabilitation efforts over a training period in which rehabilitation is performed, using image items indicating cumulative values of activity amounts of each user that are shared and displayed on the display screen.

### Functional Blocks of Rehabilitation Support System

As shown in FIG. 1, the rehabilitation support system 1 includes a data acquisition unit 10, a data collection unit 11, a data analysis unit 12, a display processing unit 13, a storage unit 14, a first display unit 15, and a second display unit 16.

The data acquisition unit 10 acquires sensor data including biological information of the users measured by the later-described sensor 105. The biological information included in the sensor data includes information relating to the beating of the heart, such as a heartbeat or a pulse measured by a wearable sensor worn by the user. Also, myogenic potential activity, blood pressure, internal gas exchanged through respiration, body temperature, pulse, and brain waves can also be used as the biological information of the users.

Also, the sensor data includes physical information of the user's body in addition to the above-described biological information. The physical information of the user's body includes acceleration indicating the user's movement, which is measured by an accelerometer. Also, the physical information of the body includes postures such as a lying position, a sitting position, and a standing position, which are estimated based on the acceleration of the user.

The data acquisition unit 10 can perform known signal processing such as noise removal and sampling of sensor data acquired chronologically, as needed.

The data collection unit 11 collects sensor data for each user acquired by the data acquisition unit 10 at regular intervals. The collected sensor data for each user is stored in the storage unit 14 together with time information.

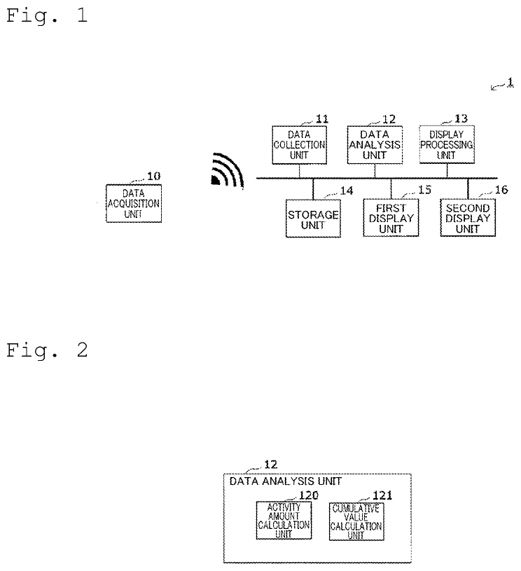

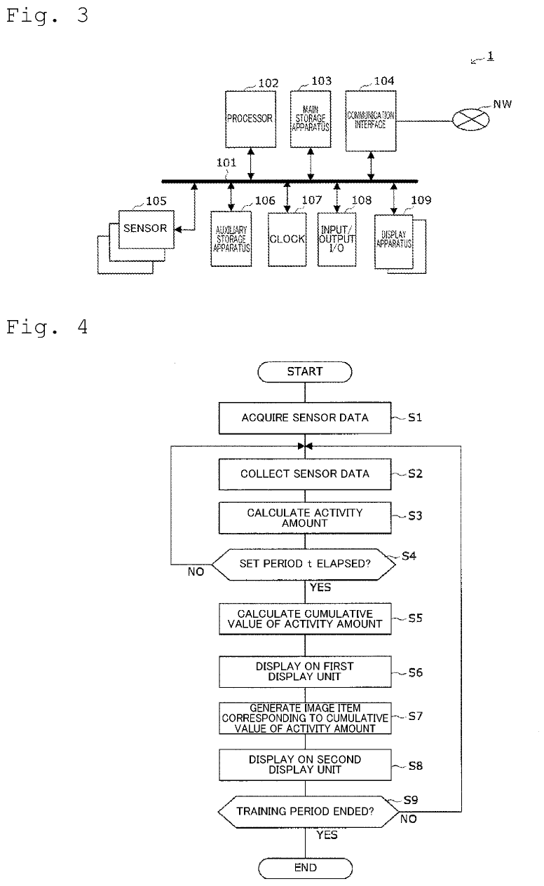

The data analysis unit 12 obtains the activity amount of each user and the cumulative value of the activity amount of each user in the set period based on the sensor data for each user collected by the data collection unit 11. As shown in FIG. 2, the data analysis unit 12 includes an activity amount calculation unit (first calculation unit) 120 and a cumulative value calculation unit (second calculation unit) 121.

The activity amount calculation unit 120 calculates the amount of physical activity accompanying the rehabilitation performed by each user based on the sensor data for each user collected by the data collection unit 11. The activity amount represents the intensity or forcefulness of the user's physical activity, and includes exercise intensity, calories burned, METs, wake-up time, standing time, step count, and the like. In the present embodiment, it is assumed that the larger the value of the user's activity amount is, the more intense the user's physical activity is, and the more actively the rehabilitation training for recovering the physical function is performed.

For example, in the case of exercise intensity, the activity amount calculation unit 120 calculates the value based on the user's heart rate acquired by the data acquisition unit 10, and the user's resting heart rate and maximum heart rate stored in advance in the storage unit 14. Also, regarding the step count, the step count of the user can be calculated based on increases/decreases in the acceleration value of the user acquired by the data acquisition unit 10.

In the case of calories burned, the activity amount calculation unit 120 calculates the calories burned based on the step count obtained based on the acceleration of the user and the movement distance. The activity amount calculation unit 120 can also calculate the calories burned with a known algorithm using the fluctuation of the heart rate in addition to the acceleration of the user. The activity amount calculation unit 120 can also convert the calories burned by the user into METs. METs are a value indicating the multiple of at-rest energy consumption that is equivalent to the energy consumption resulting from exercise.

Note that in the present embodiment, although a case is envisioned in which the target users are users who perform rehabilitation training for recovering the same motor function of the body, the activity amount calculation unit 120 can also set a weighting parameter for the value of the activity amount according to a state such as the motor function of the body of each user and calculate a relative activity amount.

The cumulative value calculation unit 121 calculates the cumulative value of the user's activity amount in the set period. For example, when a user performs rehabilitation training for 30 minutes, the cumulative value of the activity amount for each period set as appropriate, such as every 30 seconds, every minute, or every 5 minutes, can be calculated. The cumulative value of the activity amount increasing over time indicates that the user's motivation to work on rehabilitation is gradually increasing, and that the user is more actively working on rehabilitation.

For example, the cumulative value calculation unit 121 calculates a cumulative value of exercise intensity for each user, a cumulative value of calories burned, EXs (cumulative value of METs), a cumulative value of wake-up time, a cumulative value of standing time, a cumulative value of a step count, and the like.

The activity amount calculation unit 120 uses a period of the same length among a plurality of users as the set period, which is a calculation period of the cumulative value of the activity amount, but can also provide a relative calculation period according to the state of the motor function of the user's body or the like, such as the functional independence measure (FIM) of each of the plurality of users.

The display processing unit 13 generates an image item corresponding to the cumulative value of the activity amount based on the cumulative value of the activity amount of each user in the set period, which was calculated by the activity amount calculation unit 120. More specifically, the display processing unit 13 assigns any image item to each user. Image items include any still image or moving image that can be displayed in two dimensions or three dimensions, such as figures, symbols, logos, marks, numbers, text, animated characters, characters obtained by anthropomorphizing animals or other objects, and avatars.

In the present embodiment, mutually-different image items are assigned to each of the plurality of users. For example, if any figure (e.g., a triangle, a quadrilateral, a circle, etc.) is assigned to each user and the cumulative value of the user's activity amount increases over time, the number of figures assigned to the user increases.

The storage unit 14 stores the image items assigned to the users. Also, the storage unit 14 stores a function indicating the relationship between the cumulative value of the activity amount and the number of image items.

The first display unit 15 displays the cumulative value of the activity amount for each user calculated by the cumulative value calculation unit 121. The first display unit 15 can perform notification of the progress of the rehabilitation of the users to medical staff or the like managing the rehabilitation of the users by displaying the cumulative value of the activity amount for each user on the display screen. For example, the first display unit 15 can process chronological data of the cumulative value of the activity amount for each user to generate and display a graph or a table.

The second display unit 16 displays the image item generated by the display processing unit 13 on the display screen. The second display unit 16 shares and displays an image item representing the cumulative value of the activity amount of each of the plurality of users.

A user can visually recognize not only the image item corresponding to the size of the cumulative value of the user's own activity amount, which is shared and displayed on the display screen, but also the image items corresponding to the sizes of the cumulative values of the activity amounts of other users. For this reason, it is possible to relatively keep track of not only one's own rehabilitation effort but also the rehabilitation efforts of other users. For example, a user can intuitively understand whether the cumulative value of the user's own activity amount is increasing or decreasing with the passage of time compared to the cumulative value of the activity amount of another user according to an increase in the number of image items.

### Hardware Configuration of Rehabilitation Support System

Next, an example of a hardware configuration for realizing the rehabilitation support system 1 having the above-described functions will be described with reference to the block diagram of FIG. 3.

As shown in FIG. 3, the rehabilitation support system 1 can be realized by a computer including, for example, a processor 102, a main storage apparatus 103, a communication interface 104, an auxiliary storage apparatus 106, a clock 107, and an input/output I/O 108, which are connected via a bus 101, and by a program for controlling these hardware resources. The rehabilitation support system 1 includes, for example, a plurality of sensors 105 provided externally and a display apparatus 109 provided inside the rehabilitation support system 1 via the bus 101.

The main storage apparatus 103 stores programs according to which the processor 102 performs various controls and calculations, in advance. The processor 102 and the main storage apparatus 103 realize the functions of the rehabilitation support system 1 including the data analysis unit 12 and the display processing unit 13 shown in FIGS. 1 and 2.

The communication interface 104 is an interface circuit for performing communication with various external electronic devices via a communication network NW.

For example, an interface and an antenna compatible with a wireless data communication standard such as Wi-Fi (registered trademark), Bluetooth (registered trademark) Low Energy (BLE), LTE, 3G, and 5G are used as the communication interface 104. The communication interface 104 realizes the data acquisition unit 10 and the data collection unit 11 illustrated in FIG. 1.

The sensor 105 is constituted by, for example, a heart rate monitor, an electrocardiograph, a blood pressure monitor, a pulse meter, a respiratory sensor, a thermometer, a brain wave sensor, and the like. More specifically, the sensor 105 is realized by a three-axis acceleration sensor, a microwave sensor, a pressure sensor, an ammeter, a voltmeter, a thermohygrometer, a concentration sensor, a photosensor, or a combination thereof. The sensor 105 is realized as, for example, a wearable sensor. Also, the sensor 105 realizes the data acquisition unit 10 illustrated in FIG. 1.

The auxiliary storage apparatus 106 is constituted by a readable and writable storage medium and a drive apparatus for reading and writing various types of information such as programs and data from and to the storage medium. A hard disk or a semiconductor memory such as a flash memory can be used as the storage medium in the auxiliary storage apparatus 106.

The auxiliary storage apparatus 106 has a storage region for storing biological information such as the heartbeat measured by the sensor 105 and physical information of the body such as the acceleration, and a program storage area for storing a program according to which the rehabilitation support system 1 performs analysis processing for sensor data. The auxiliary storage apparatus 106 realizes the storage unit 14 illustrated in FIG. 1. Furthermore, for example, the auxiliary storage apparatus 106 may also have a backup region or the like for backing up the above-described data, programs, and the like.

The clock 107 is constituted by a built-in clock or the like built in a computer, and measures the time. Alternatively, the clock 107 may also acquire time information from a time server (not shown). The time information obtained by the clock 107 is used for the timing at which the data collection unit 11 periodically collects the sensor data, the calculation period during calculation of the cumulative value of the activity amount for each period set by the cumulative value calculation unit 121, the sampling of the sensor data, and the like.

The input/output I/O 108 is constituted by an I/O terminal that accepts input of a signal from an external device such as a sensor 105 or a display apparatus 109, and outputs a signal to the external device.

The display apparatus 109 is realized by a liquid crystal display or the like. The display apparatus 109 realizes the first display unit 15 illustrated in FIG. 1 and the second display unit 16 of the shared monitor. The display apparatus 109 that realizes the second display unit 16 of the shared monitor is realized by one display apparatus 109 installed in, for example, a rehabilitation training room, but it is also possible to employ a configuration in which a plurality of display apparatuses 109 are used, as long as shared display is possible.

Note that the rehabilitation support system 1 is not limited to the case of being realized by a single computer, and each function of the rehabilitation support system 1 shown in FIGS. 1 and 2 can be distributed by a plurality of computers on the communication network NW.

### Operation of Rehabilitation Support System

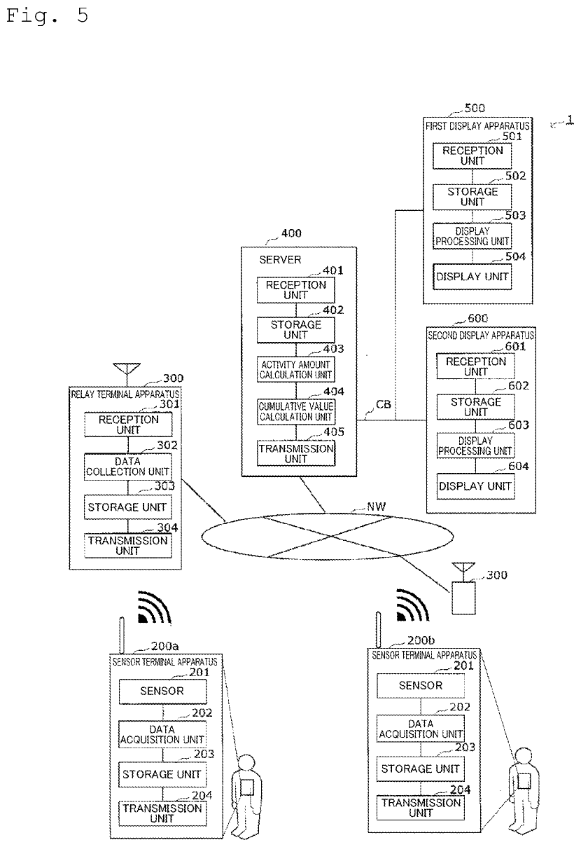

Next, operations of the rehabilitation support system 1 having the above-described configuration will be described with reference to the flowchart of FIG. 4. The plurality of users performing rehabilitation put on sensors 105 (data acquisition units 10) constituted by wearable sensors in advance and start rehabilitation training. Also, hereinafter, for the sake of simplicity in the description, a case will be described in which a plurality of users start rehabilitation training at the same time and perform training until the end of a pre-set training period.

First, the data acquisition unit 10 acquires sensor data including biological information and physical information of the body measured by the sensor 105 for each user (step S1). For example, the data acquisition unit 10 acquires the user's heartbeat and acceleration as the sensor data.

Next, the data collection unit 11 collects the sensor data of a plurality of users from the data acquisition unit 10 at regular intervals (step S2). Thereafter, the activity amount calculation unit 120 calculates the activity amount for each user based on the collected sensor data (step S3). The activity amount calculation unit 120 calculates, for example, exercise intensity, calories burned, wake-up time, standing time, step count, and the like for each user as the activity amount. The calculated chronological data of the activity amount for each user is accumulated in the storage unit 14.

Next, for example, when the set period t elapses from the start of the calculation of the activity amount (step S4: YES), the cumulative value calculation unit 121 calculates the cumulative value of the activity amount for each user (step S5). On the other hand, if the set period t has not elapsed (step S4: NO), steps S2 and S3 are repeated.

Next, the first display unit 15 displays the cumulative value of the activity amount for each user upon the elapse of the set period t calculated in step S5 on, for example, the display screen (step S6). The data of the cumulative value of the activity amount displayed by the first display unit 15 is used by the medical staff managing the rehabilitation of the users, or the like. Accordingly, data processed into a graph, a table, or the like is used as the data displayed on the display screen.

Next, the display processing unit 13 generates a number of image items corresponding to the cumulative values of the activity amounts based on the cumulative values of the activity amounts of the users upon the elapse of the set period t calculated in step S5 (step S7). For example, the display processing unit 13 assigns a mark of a figure (triangle, quadrilateral, circle, etc.) to each user, generates a larger number of figures the greater the cumulative value of the activity amount is, and generates a smaller number of figures the smaller the cumulative value of the activity amount is. In this manner, the cumulative values of the activity amounts of the plurality of users are represented by image items according to which the sizes or the increases of the cumulative values can be easily visually understood based on a common standard.

Next, the second display unit 16 displays the image item for each user generated by the display processing unit 13 on the display screen (step S8). For example, the second display unit 16 can display image items of each of the plurality of users in the same region of the shared monitor installed in the training room where the plurality of users are performing rehabilitation training. For example, the second display unit 16 displays figures (triangles, quadrilaterals, circles, etc.) assigned to each of a plurality of users on the display screen, the number of the figures corresponding to the cumulative value of the activity amount of each user.

Thereafter, the processing from step S2 to step S8 is executed repeatedly until the period of rehabilitation training ends (step S9: NO). The second display unit 16 updates the image item of each user according to the size of the cumulative value of the user's activity amount and shares and displays the image item on the display screen every time the set period t elapses.

For example, the second display unit 16 updates the number of figures assigned to each user and displays them on the display screen each time the set period t elapses. For this reason, the user can relatively understand the rehabilitation efforts of the user and other users according to the number of each figure, which changes over time.

### Specific Configuration Example of Rehabilitation Support System

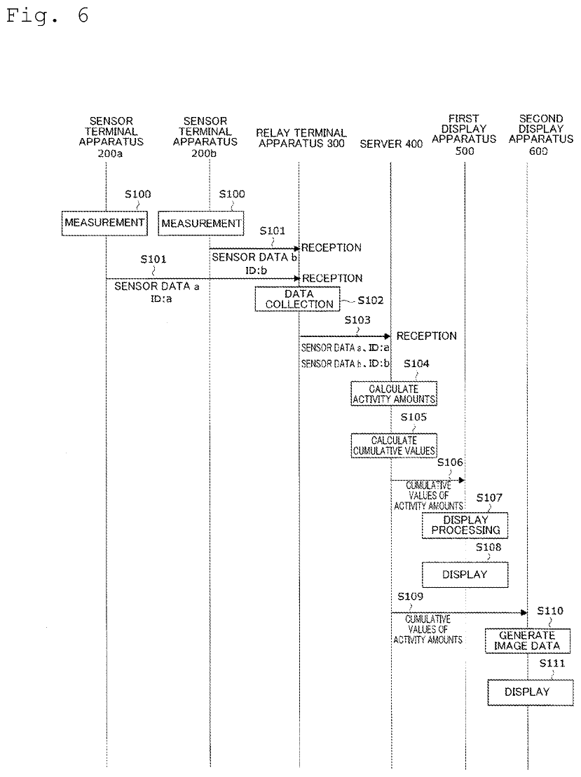

Next, a specific configuration example of the rehabilitation support system 1 having the above-described configuration will be described with reference to the block diagram of FIG. 5.

As shown in FIG. 5, for example, the rehabilitation support system 1 includes sensor terminal apparatuses 200a and 200b that are worn by the users performing rehabilitation, a relay terminal apparatus 300, a server 400, a first display apparatus 500, and a second display apparatus 600. All or any of the sensor terminal apparatuses 200a and 200b, the relay terminal apparatus 300, the server 400, the first display apparatus 500, and the second display apparatus 600 include the functions included in the rehabilitation support system 1, such as the data analysis unit 12 illustrated in FIGS. 1 and 2.

Note that hereinafter, the sensor terminal apparatuses 200a and 200b include the data acquisition unit 10 illustrated in FIG. 1, and the relay terminal apparatus 300 includes the data collection unit 11. Also, a case will be described in which the server 400 includes the data analysis unit 12, the first display apparatus 500 includes the first display unit 15, and furthermore, the second display apparatus 600 includes the display processing unit 13 and the second display unit 16.

### Functional Blocks of Sensor Terminal Apparatuses

As shown in FIG. 5, the sensor terminal apparatuses 200a and 200b each include a sensor 201, a data acquisition unit 202, a storage unit 203, and a transmission unit 204. The sensor terminal apparatuses 200a and 200b are arranged on the trunks or the like of the users, for example, and are worn on the users to measure sensor data including biological information and physical information of the body. The sensor terminal apparatuses 200a and 200b transmit the measured biological information such as the heartbeat or the pulse and physical information of the body such as acceleration or posture of the users to the relay terminal apparatus 300 through wireless communication. For example, wristwatch-type or clothing-type wearable terminals are used as the sensor terminal apparatuses 200a and 200b.

The sensor 201 is realized by, for example, a three-axis acceleration sensor or an electrocardiograph. Regarding the three axes of the acceleration sensor, for example, the X-axis is provided parallel to the left-right direction of the body, the Y-axis is provided parallel to the front-rear direction of the body, and the Z-axis is provided parallel to the vertical direction of the body.

The data acquisition unit 202 acquires the sensor data measured by the sensor 201, and performs known signal processing such as noise removal and sampling processing as needed.

The storage unit 203 accumulates the chronological sensor data acquired by the data acquisition unit 202.

The transmission unit 204 sends the sensor data including the biological information of the users acquired by the data acquisition unit 202 to the relay terminal apparatus 300. The transmission unit 204 can transmit the sensor data to the relay terminal apparatus 300 through wireless communication such as BLE or Wi-Fi, for example.

### Functional Blocks of Relay Terminal Apparatus

A plurality of relay terminal apparatuses 300 are installed in a predetermined region such as a hospital or a rehabilitation facility, and each has its own communication area. The relay terminal apparatuses 300 transmit data to the server 400 via the communication network NW. As shown in FIG. 5, the relay terminal apparatus 300 includes a reception unit 301, a data collection unit 302, a storage unit 303, and a transmission unit 304.

The reception unit 301 receives the sensor data including the biological information of the user and the physical information of the body acquired by the sensor terminal apparatuses 200a and 200b.

The data collection unit 302 collects the received sensor data for each user at regular intervals.

The storage unit 303 stores the collected sensor data for each user.

The transmission unit 304 transmits the sensor data for each user collected by the data collection unit 302 at regular intervals to the server 400 via the communication network NW.

### Functional Blocks of Server

The server 400 includes a reception unit 401, a storage unit 402, an activity amount calculation unit 403, a cumulative value calculation unit 404, and a transmission unit 405. The server 400 calculates the activity amount of each user and the cumulative value of the activity amount based on the sensor data collected in the relay terminal apparatus 300.

The reception unit 401 receives the sensor data from the relay terminal apparatus 300 via the communication network NW.

The storage unit 402 accumulates the received sensor data for each user.

The activity amount calculation unit 403 calculates the activity amount based on the user's sensor data.

The cumulative value calculation unit 404 calculates the cumulative value of the user's activity amount in the set period.

The transmission unit 405 sends, for example, the cumulative value of the user's activity amount to the first display apparatus 500 and the second display apparatus 600 connected by a cable CB.

### Functional Blocks of First Display Apparatus

The first display apparatus 500 corresponds to the first display unit 15 illustrated in FIG. 1. The first display apparatus 500 is connected to the server 400 by, for example, the cable CB.

The first display apparatus 500 includes a reception unit 501, a storage unit 502, a display processing unit 503, and a display unit 504.

The reception unit 501 receives the cumulative value of the activity amount for each user calculated by the cumulative value calculation unit 404.

The storage unit 502 stores the received cumulative value of the activity amount for each user.

The display processing unit 503 generates a graph or a table based on the chronological data of the cumulative value of the activity amount for each user.

The display unit 504 displays the graph or table showing chronological data of the cumulative value of the activity amount for each user on the display screen.

### Functional Blocks of Second Display Apparatus

The second display apparatus 600 corresponds to the second display unit 16 illustrated in FIG. 1. The second display apparatus 600 is connected to the server 400 via, for example, the cable CB.

The second display apparatus 600 includes a reception unit 601, a storage unit 602, a display processing unit 603, and a display unit 604.

The reception unit 601 receives the cumulative value of the activity amount for each user calculated by the cumulative value calculation unit 404 via the cable CB.

The storage unit 602 stores the received cumulative value of the activity amount for each user.

The display processing unit 603 corresponds to the display processing unit 13 illustrated in FIG. 1 and generates an image item corresponding to the cumulative value of the user's activity amount every time the set period elapses. The display processing unit 603 assigns image items such as figures and characters that are different from each other to each user, and generates a number of image items corresponding to the size of the cumulative value of the user's activity amount. The display processing unit 603 generates an image item every time the set period elapses.

The display unit 604 displays the image items generated by the display processing unit 603 on the display screen, which is a shared monitor. The display unit 604 can update and display the number of image items every time the set period elapses until the period in which the user performs the rehabilitation training ends.

### Operation Sequence of Rehabilitation Support System

Next, operations of the rehabilitation support system 1 according to the specific configuration illustrated in FIG. 5 will be described with reference to the sequence diagram of FIG. 6. Hereinafter, for the sake of simplicity in the description, a case will be described in which two users put on the sensor terminal apparatuses 200a and 200b and start rehabilitation training.

First, the sensor 201 measures biological information such as the heartbeat or pulse of the user performing rehabilitation training with the sensor terminal apparatuses 200a and 200b, and physical information of the body such as the acceleration and posture (step S100). Next, the sensor terminal apparatuses 200a and 200b transmit sensor data a and b that were measured by the sensor 201 and were thereafter acquired by the data acquisition unit 202 to the relay terminal apparatus 300 via BLE or Wi-Fi (step S101). Note that as shown in FIG. 6, each piece of sensor data includes information (ID: a, ID: b) for identifying the user.

For example, the relay terminal apparatus 300 collects the sensor data acquired by the sensor terminal apparatuses 200a and 200b at regular intervals (step S102). Next, the relay terminal apparatus 300 transmits the collected sensor data of the plurality of users to the server 400 via the communication network NW (step S103).

Next, the server 400 calculates the activity amount for each user based on the received sensor data of the user (step S104). Next, the server 400 calculates the cumulative value of the activity amount for each user in the set period (step S105). Thereafter, the server 400 sends, for example, the cumulative value of the activity amount for each user calculated in step S105 to the first display apparatus 500, which is used by the medical staff and is connected by the cable CB (step S106).

After that, the first display apparatus 500 generates a graph or a table based on the chronological data of the cumulative value of the activity amount for each user (step S107). Next, the first display apparatus 500 displays the generated graph or table on the display screen (step S108). For example, as shown in FIG. 13, the first display apparatus 500 (first display unit 15) can generate and display a table of cumulative values of the activity amounts for each user.

On the other hand, the server 400 sends the cumulative value of the user's activity amount upon the elapse of the set period calculated in step S105 to, for example, the second display apparatus 600 connected via the cable CB (step S109).

The display processing unit 603 of the second display apparatus 600 generates an image item corresponding to the cumulative value of the user's activity amount (step S110). For example, the display processing unit 603 can assign mutually-different image items such as any figure to each user, and can generate a larger number of figures the larger the cumulative value of the activity amount is.

Next, the second display apparatus 600 displays image items corresponding to the generated cumulative values of the activity amounts of the plurality of users on the display screen, which is the shared monitor (step S111).

### First Display Example

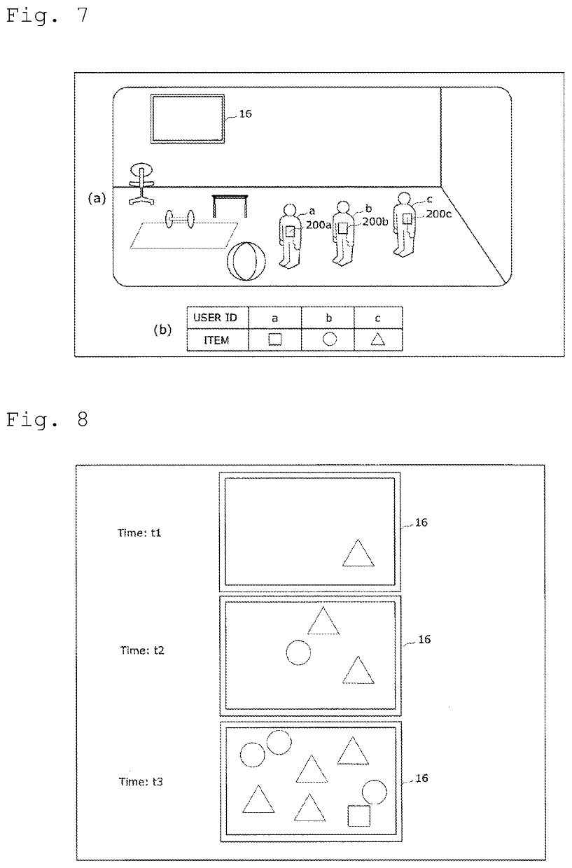

Next, display processing performed by the rehabilitation support system 1 according to the present embodiment and a data display example will be described. FIGS. 7 and 8 are diagrams for illustrating a first display example performed by the rehabilitation support system 1. FIG. 7(a) shows an example in which a plurality of users a, b, and c performing rehabilitation for restoring motor function of the body perform rehabilitation training in the training room. As shown in FIG. 7(a), in the training room, the second display unit 16 is installed as a shared monitor at a position visible to all of the plurality of users a, b, and c performing rehabilitation.

FIG. 7(b) shows mutually-different image items assigned as appropriate to users a, b, and c. In FIG. 7(b), user a is assigned a “quadrilateral” mark, user b is assigned a “circle” mark, and user c is assigned a “triangle” mark.

FIG. 8 shows a state in which the second display unit 16 updates the number of image items over time and displays them on the display screen. In FIG. 8, time elapses in the order of times t1, t2, and t3. At time t1, the “triangle” mark indicating the cumulative value of the activity amount of the user c is displayed on the display screen, but the marks indicating the cumulative values of the activity amounts of the other users a and b are not displayed. At time t1, it is shown that the activity amount of user c is relatively high, and that the rehabilitation of user c is more advanced than that of other users.

Thereafter, at time t2, the number of “triangle” marks of user c is further increased, and the “circle” mark assigned to user b is newly displayed on the display screen. At time t2, it is shown that rehabilitation is being performed more actively in the order of user c, user b, and user a.

After that, at time t3, the number of “triangle” marks of user c further increases. Also, the number of “circle” marks of user b has also increased, and a “quadrilateral” mark of user a is newly displayed. Users a, b, and c each work on rehabilitation training individually, but the progress and efforts of rehabilitation of not only themselves but also other users can be understood relatively due to the increases in the number of image items displayed on the display screen. For this reason, it is possible to improve the motivation of all users to work on rehabilitation, including the user a, who has a relatively small activity amount. In particular, sharing the increases in the activity amounts of the user c and the user b who have a relatively large activity amount with the user a who has a relatively small activity amount has a positive effect on the user a, and thus it is possible to motivate the user a to work on rehabilitation.

### Second Display Example

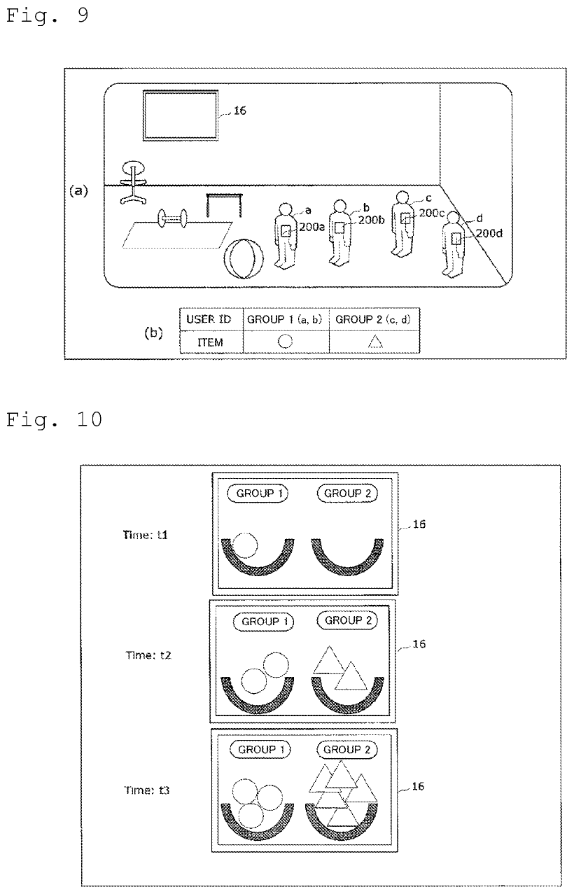

FIG. 9 and 10 are diagrams for describing another example of display processing performed by the rehabilitation support system 1. As shown in FIG. 9(a), in a second display example, four users a, b, c, and d are divided into two groups, and an image item is assigned to each group. As shown in FIG. 9(b), users a and b are grouped into “group 1” to which the “circle” mark is assigned, and users c and d are grouped into “group 2” to which the “triangle” mark is assigned.

As shown in FIG. 10, image items corresponding to the sizes of the cumulative values of the activity amounts are shared and displayed on the display screen for each of the groups 1 and 2 in which the plurality of users a, b, c, and d are grouped. Also, in the example of FIG. 10, a container such as a basket for each group is displayed in advance on the background of the display screen, and an image item is displayed as being put in the container of each group. In this manner, the display screen displays how the number of image items in the basket increases according to the progress and effort of rehabilitation for each group.

At time t1, an image in which one “circle” mark is displayed in the basket of group 1 and nothing is in the basket of group 2 is displayed. At time t1, through comparison of the number of image items, it is understood that the total number of cumulative values of activity amounts calculated accompanying the rehabilitation performed by “group 1” of the users a and b is greater than the total number of cumulative values of activity amounts of “group 2” of the users c and d.

At time t2, the number of “circle” marks in the group 1 basket has increased to two, and two new “triangle” marks are being displayed in the other basket of group 2. Accordingly, at time t2, it is displayed that the cumulative values of the activity amounts of group 1 and group 2 are equal. It can also be said that the users c and d of group 2, who were relatively unmotivated to perform rehabilitation at time t1, were influenced by the active efforts for rehabilitation of group 1 at time t1, and their motivation improved thereafter.

At time t3, a state is displayed on the display screen in which one “circle” mark is added to the group 1 basket to make a total of three, and three new “triangle” marks are added to the group 2 basket to make a total of five. At time t3, it is understood from the number of image items that users c and d in group 2 are more actively performing rehabilitation compared to group 1.

In this manner, the second display unit 16 displays image items corresponding to the size of the cumulative value of the activity amount in each group in which a plurality of users are grouped on a display screen such as a shared monitor, and thereby it is possible to improve the activity amount of all users by increasing competitive rivalry between the groups.

Note that a plurality of users can be grouped as appropriate to form the groups, but for example, they can be grouped based on various physical factors such as age and gender. Also, the number of people in each group is not limited to being the same number.

When image items are generated according to the size of the cumulative value of the activity amount in units of groups as in this second display example, the cumulative value calculation unit 121 and the display processing unit 13 operate as follows.

The cumulative value calculation unit 121 calculates the cumulative value of the activity amount for each group each time the set period elapses, based on the activity amount for each user calculated by the activity amount calculation unit 120. For example, the cumulative value calculation unit 121 can calculate the cumulative value of the activity amount of each of the users a and b, and calculate the sum of them as the cumulative value of the activity amount of the group 1.

The display processing unit 13 generates image items according to the size of the cumulative value of the activity amount based on the cumulative value of the activity amount for each group calculated by the cumulative value calculation unit 121. For example, as illustrated in the above-described second display example, it is possible to use a configuration in which image items such as figures are assigned to each group and the number of image items increases as the cumulative value of the activity amount for each group increases.

### Third Display Example

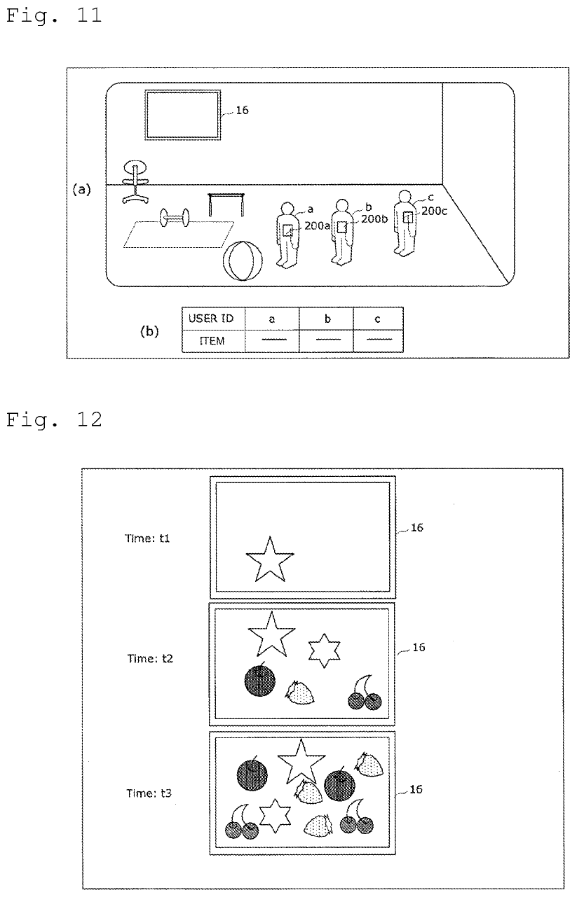

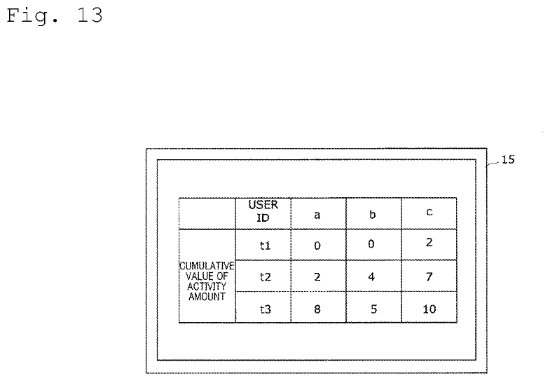

Next, another example of display processing performed by the rehabilitation support system 1 will be described with reference to FIGS. 11 and 12. As shown in FIG. 11(a), three users a, b, and c perform rehabilitation in the training room. In this third display example, there is no individual image item assigned to each user (FIG. 11(b)), but common image items are assigned to all users.

As shown in FIG. 12, image items corresponding to the size of the sum of the cumulative values of the activity amounts of all the users a, b, and c are displayed on the shared monitor. For example, at time t1, one “star” is displayed on the display screen as an image item corresponding to the size of the sum of the cumulative values of the activity amounts of all the users a, b, and c.

Thereafter, at time t2, the sum of the cumulative values of the activity amounts of users a, b, and c increases, and in addition to the “star” image item, “apple”, “strawberry”, and “cherry” image items are newly displayed on the display screen.

Then, at time t3, the number of “apple”, “strawberry”, and “cherry” image items further increases, and a large number of image items are displayed so as to cover the entire region of the display screen.

In this manner, in the third display example, image items for all users corresponding to the size of the sum of the temporal cumulative values of the activity amounts accompanying performance of rehabilitation of the plurality of users are shared and displayed, and therefore it is possible to improve the motivation for rehabilitation even for a user with a relatively small activity amount by sharing the increases in the cumulative values of the activity amounts of all of the users. Also, by sharing and displaying an image item indicating the sum of the cumulative values of the activity amounts of all users, there is a synergistic effect of improving motivation for mutual rehabilitation among users.

Note that in the third display example, a case was illustrated in which the sum of the cumulative values of the activity amounts of all the users is indicated by the number of any different image items, but it is also possible to generate and display any identical image items for all of the users.

As described above, with the rehabilitation support system 1 according to the present embodiment, the cumulative value of the activity amount of each user or a plurality of users is calculated each time the set period elapses, and an image item corresponding to the cumulative value of the activity amount is generated, and is displayed on a display screen, which is a shared monitor. For this reason, a user performing rehabilitation can relatively understand his or her own effort toward rehabilitation in relation to the rehabilitation effort of another user. In particular, an increase in the activity amount of other users can be shared even to a user with a relatively small activity amount, and thus motivation for rehabilitation can be improved for that user.

Also, in the rehabilitation support system 1 according to the present embodiment, since the cumulative values of the activity amounts of a plurality of users are shared and displayed, it is possible to improve the motivation for the rehabilitation of all of the users.

Also, in the rehabilitation support system 1 according to the present embodiment, the number of displayed image items increases according to the temporal increase in the cumulative value of the user's activity amount. For this reason, even if a user is concentrating on rehabilitation, the user can intuitively understand an increase in the activity amount of the user or another user by merely glancing at an image displayed on the shared monitor.

Also, the user can perform rehabilitation more enjoyably by performing rehabilitation while observing how image items such as characters familiar to the user, which are displayed on the shared monitor, change.

Note that in the above-described embodiment, a case was described in which a still image in particular is displayed on the display screen as the image item, but the image item may also be a moving image. For example, it is also possible to use a mode in which image items such as figures as described in the first to third display examples start moving accompanying an increase in the cumulative value of the activity amount. In addition, as long as the user can understand the increase in the activity amounts of a plurality of users at a glance while performing rehabilitation, it is also possible to display not only an increase in the number of image items, but also a state in which the surface area occupied by an image item on a display screen increases, or a state in which the color of an image item is gradually filled in from a state of being transparent. Alternatively, it is also possible to display an image item that imitates a process of building and completing a building.

The image item can be not only a two-dimensional image, but also, for example, a three-dimensional image such as a hologram projected on a spatially shared monitor.

The display processing unit 13 may also use a configuration in which audio data to be added to the image item is generated and sound is output from a speaker (not shown) when the image item is displayed on the display screen.

Also, in the above-described embodiment, as described in first to third display examples, a case was described in which the second display unit 16 is installed as one shared monitor in a training room where a plurality of users perform rehabilitation. However, the second display unit 16 need only be able to share and display the image items generated by the display processing unit 13, and for example, a shared monitor may also be installed in each of a plurality of training rooms. Alternatively, image items corresponding to the cumulative values of the activity amounts of a plurality of users can be shared and displayed on a display screen of a smartphone or the like carried by each user.

Although embodiments of the rehabilitation support system and the rehabilitation support method of the present invention have been described above, the present invention is not limited to the above-described embodiments, and it is possible to make various modifications that can be envisioned by a person skilled in the art in the scope of the invention described in the claims.

### REFERENCE SIGNS LIST

1 Rehabilitation support system
10, 202 Data acquisition unit
11, 302 Data collection unit
12 Data analysis unit
13, 503, 603 Display processing unit
14, 203, 303, 402, 502, 602 Storage unit
15 First display unit
16 Second display unit
120, 403 Activity amount calculation unit
121, 404 Cumulative value calculation unit
101 Bus
102 Processor
103 Main storage apparatus
104 Communication interface
105 Sensor
106 Auxiliary storage apparatus
107 Clock
108 Input/output I/O
109 Display apparatus
200, 200a, 200b, 200c, 200d Sensor terminal apparatus
300 Relay terminal apparatus
400 Server
500 First display apparatus
600 Second display apparatus
204, 304, 405 Transmission unit
301, 401, 501, 601 Reception unit

1-8. (canceled)

9. A rehabilitation support system comprising:
a data acquirer configured to acquire sensor data comprising biological information;
a first calculator configured to calculate an activity amount of a user based on the sensor data;
a second calculator configured to calculate a cumulative value of the activity amount of the user in a set period, wherein a period of activity of the user comprises a plurality of the set periods;
a display processor configured to generate an image item corresponding to the cumulative value; and
a display configured to display, on a display screen, the image item generated by the display processor.

10. The rehabilitation support system according to claim 9, wherein the display processor is configured to generate a number of the image items corresponding to the cumulative value each time the cumulative value is calculated by the second calculator.

11. The rehabilitation support system according to claim 9, wherein:
the data acquirer is configured to acquire the sensor data of each of a plurality of users;
the first calculator is configured to calculate an activity amount of each of the plurality of users based on the sensor data;
the second calculator is configured to calculate a cumulative value of the activity amount of each of the plurality of users; and
the display processor is configured to display, for each of the users, the image item corresponding to the cumulative value of each of the plurality of users that was calculated by the second calculator.

12. The rehabilitation support system according to claim 9, wherein:
the data acquirer is configured to acquire the sensor data of each of a plurality of users grouped into groups in advance;
the second calculator is configured to calculate a cumulative value of the activity amount in the set period for each group into which the plurality of users were grouped in advance;
the display processor is configured to generate the image item corresponding to the cumulative value for each group; and
the display is configured to display on the display screen the image item for each group that was generated by the display processor.

13. The rehabilitation support system according to claim 9, wherein:
the display comprises at least one display apparatus having the display screen; and
the display apparatus is configured to display the image item generated by the display processor on the display screen.

14. The rehabilitation support system according to claim 9, wherein the image item comprises a figure, a character, or an avatar.

15. The rehabilitation support system according to claim 9, wherein:
the biological information comprises information indicating beating of a heart measured by a wearable sensor worn by the user; and
the sensor data further comprises acceleration information indicating a movement of the user measured by an acceleration sensor.

16. A rehabilitation support method comprising:
acquiring sensor data comprising biological information of a user measured by a sensor;
calculating an activity amount of a user based on the sensor data;
calculating a cumulative value of the activity amount of the user in a set period, wherein a period of activity of the user comprises a plurality of the set periods;
generating an image item corresponding to the cumulative value; and
displaying, on a display screen, the image item.

17. The rehabilitation support method according to claim 16, wherein generating the image item further comprises generating a number of the image items corresponding to the cumulative value each time the cumulative value is calculated.

18. The rehabilitation support method according to claim 16, further comprising:
acquiring the sensor data of each of a plurality of users;
calculating an activity amount of each of the plurality of users based on the sensor data;
calculating a cumulative value of the activity amount of each of the plurality of users; and
displaying, for each of the users, the image item corresponding to the cumulative value of each of the plurality of users.

19. The rehabilitation support method according to claim 16, further comprising:
acquiring the sensor data of each of a plurality of users grouped into groups in advance;
calculating a cumulative value of the activity amount in the set period for each group into which the plurality of users were grouped in advance;
generating the image item corresponding to the cumulative value for each group; and
displaying on the display screen the image item for each group.

20. The rehabilitation support method according to claim 16, further comprising at least one display apparatus having the display screen, wherein the display apparatus displays the image item on the display screen.

21. The rehabilitation support method according to claim 16, wherein the image item comprises a figure, a character, or an avatar.

22. The rehabilitation support method according to claim 16, wherein:
the biological information comprises information indicating beating of a heart measured by a wearable sensor worn by the user; and
the sensor data further comprises acceleration information indicating a movement of the user measured by an acceleration sensor.
